(19) United States
(12) Patent Application Publication
(10) Pub. No.: US 2025/0228654 A1
(43) Pub. Date: Jul. 17, 2025

(54) MULTI-FUNCTION SURGICAL LOCKING BUTTONS

(71) Applicant: ARTHREX, INC., Naples, FL (US)

(72) Inventors: Peter J. Dreyfuss, Naples, FL (US); Benedikt Beyer, Germering (DE); Justin Boyle, Naples, FL (US); Jacob A. Jolly, Naples, FL (US)

(21) Appl. No.: 18/420,072

(22) Filed: Jan. 23, 2024

Related U.S. Application Data
(60) Provisional application No. 63/619,831, filed on Jan. 11, 2024.

(51) Int. Cl. A61F 2/08

(52) U.S. Cl. CPC A61F 2/0811; A61F 2002/0829; A61F 2002/0852; A61F 2002/0858; A61F 2002/0882

(57) ABSTRACT

Graft fixation systems and methods are provided for securing a graft within a bone tunnel. An exemplary graft fixation system may include a surgical locking button adapted for fixating both a graft and a reinforcement suture relative to bone.

### CROSS-REFERENCE TO RELATED APPLICATIONS

This application claims the benefit of U.S. Provisional Application No. 63/619,831, which was filed on Jan. 11, 2024 and is incorporated herein by reference in its entirety.

### BACKGROUND

This disclosure relates to the field of surgery, and more particularly to graft fixation systems that include a surgical locking button adapted to establish a common point of fixation for a graft and a reinforcement suture that is separate from the graft.

Tissue reconstruction surgeries, such as anterior cruciate ligament (ACL) reconstructions and posterior cruciate ligament (PCL) reconstructions, typically involve drilling a tunnel through bone, positioning a substitute graft in the bone tunnel, and fixating the graft within the bone tunnel using fixation devices, such as buttons, screws, or the like.

### SUMMARY

This disclosure relates to graft fixation systems and methods for securing a graft within a bone tunnel. The graft fixation system can be used in various tissue reconstruction procedures, including but not limited to, ACL and PCL reconstructions, for example.

An exemplary surgical locking button may include, inter alia, a disk portion and a collar portion protruding from the disk portion. A proximal surface of the collar portion establishes a bridge for securing a graft relative to a bone. A screw or a door is connectable to the disk portion and is configured for locking a reinforcement suture relative to the bone.

Another exemplary surgical locking button may include, inter alia, a disk portion including a threaded opening, and an eyelet connectable to the disk portion. The eyelet includes a threaded portion that is configured to engage the threaded opening. The eyelet is further configured to secure both a graft and a reinforcement suture relative to a bone.

An exemplary graft fixation system may include, inter alia, a surgical locking button including a disk portion that includes a top surface and a bottom surface. A slot bisects the disk portion, and a bridge extends across the slot. A suture loop is looped over the bridge. A reinforcement suture extends through the disk portion and under a loop section of the suture loop to trap the reinforcement suture between the loop section and the bridge.

An exemplary surgical method may include, inter alia, positioning a surgical locking button relative to a bone tunnel of a bone. The surgical locking button fixates both a graft and a reinforcement suture relative to the bone.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a graft fixation system for securing a graft within a bone tunnel.

FIGS. 2 and 3 illustrate an exemplary surgical locking button of the graft fixation system of FIG. 1.

FIGS. 4, 5, and 6 illustrate another exemplary surgical locking button.

FIGS. 7, 8, and 9 illustrate another exemplary surgical locking button.

FIGS. 10, 11, and 12 illustrate another exemplary surgical locking button.

FIG. 13 illustrates another exemplary surgical locking button.

FIGS. 14, 15, and 16 illustrate another exemplary surgical locking button.

FIGS. 17 and 18 illustrate another exemplary surgical locking button.

FIGS. 19, 20, and 21 illustrate another exemplary surgical locking button.

FIG. 22 illustrates another exemplary surgical locking button.

FIGS. 23, 24, and 25 illustrate yet another exemplary surgical locking button.

FIG. 26 illustrates yet another exemplary surgical locking button.

FIG. 27 illustrates yet another exemplary surgical locking button.

### DETAILED DESCRIPTION

This disclosure relates to graft fixation systems and methods for securing a graft and a reinforcement suture that is separate from the graft within a bone tunnel. The graft fixation system can be used in various tissue reconstruction procedures, including but not limited to, ACL and PCL reconstructions, for example. These and other features of this disclosure are described in further detail below.

An exemplary surgical locking button may include, inter alia, a disk portion and a collar portion protruding from the disk portion. A proximal surface of the collar portion establishes a bridge for securing a graft relative to a bone. A screw or a door is connectable to the disk portion and is configured for locking a reinforcement suture relative to the bone.

In any further embodiment, the disk portion and the collar portion establish a unitary body of the surgical locking button.

In any further embodiment, a slot bisects the disk portion.

In any further embodiment, a threaded opening is formed in the slot and is configured to receive the screw for trapping the reinforcement suture between the screw and the bridge.

In any further embodiment, a threaded opening is formed in the disk portion at a location that is offset from the slot. The threaded opening is configured to receive the screw for trapping the reinforcement suture between the screw and a floor of a suture tunnel that extends beneath the threaded opening.

In any further embodiment, a threaded opening is formed in the disk portion at a location that is offset from the slot. The threaded opening is configured to receive the screw for trapping the reinforcement suture between the screw and a floor of a second slot that bisects the threaded opening.

In any further embodiment, a threaded opening is formed in the disk portion at a location that is offset from the slot. The threaded opening is configured to receive the screw for trapping the reinforcement suture between a first threading of the screw and a second threading of the threaded opening.

In any further embodiment, a threaded opening is formed in the disk portion at a location that is offset from the slot. The threaded opening is configured to receive the screw for trapping the reinforcement suture between an eyelet of the screw and the threaded opening.

In any further embodiment, an unthreaded opening is formed in the disk portion at a location that is offset from the slot. The unthreaded opening is configured to receive the screw for trapping the reinforcement suture between an eyelet of the screw and the unthreaded opening.

In any further embodiment, the screw is secured within the unthreaded opening by a screw nut.

In any further embodiment, a through passage is formed in the disk portion at a location that is offset from the slot. The door is pivotably mounted within the through passage by a cross-pin.

In any further embodiment, the door includes a first set of teeth configured to engage a second set of teeth of the disk portion for locking the reinforcement suture relative to the disk portion.

Another exemplary surgical locking button may include, inter alia, a disk portion including a threaded opening, and an eyelet connectable to the disk portion. The eyelet includes a threaded portion that is configured to engage the threaded opening. The eyelet is further configured to secure both a graft and a reinforcement suture relative to a bone.

In any further embodiment, a through-hole of the eyelet is configured to receive a suture loop for securing the graft relative to the bone. The threaded opening is configured to receive the threaded portion for trapping the reinforcement suture between a first threading of the eyelet and a second threading of the threaded opening.

In any further embodiment, a suture loop connected to the graft and the reinforcement suture are clamped between a pin of the disk portion and a bridge of the eyelet when the eyelet is connected to the disk portion.

An exemplary graft fixation system may include, inter alia, a surgical locking button including a disk portion that includes a top surface and a bottom surface. A slot bisects the disk portion, and a bridge extends across the slot. A suture loop is looped over the bridge. A reinforcement suture extends through the disk portion and under a loop section of the suture loop to trap the reinforcement suture between the loop section and the bridge.

An exemplary surgical method may include, inter alia, positioning a surgical locking button relative to a bone tunnel of a bone. The surgical locking button fixates both a graft and a reinforcement suture relative to the bone.

In any further embodiment, the graft is secured to the surgical locking button by a suture or a suture loop.

In any further embodiment, a screw or a door of the surgical locking button locks a position of the reinforcement suture relative to the surgical locking button.

In any further embodiment, an eyelet of the surgical locking button locks a position of the reinforcement suture relative to the surgical locking button.

In any further embodiment, a suture loop that connects the graft to the surgical locking button locks a position of the reinforcement suture relative to the surgical locking button.

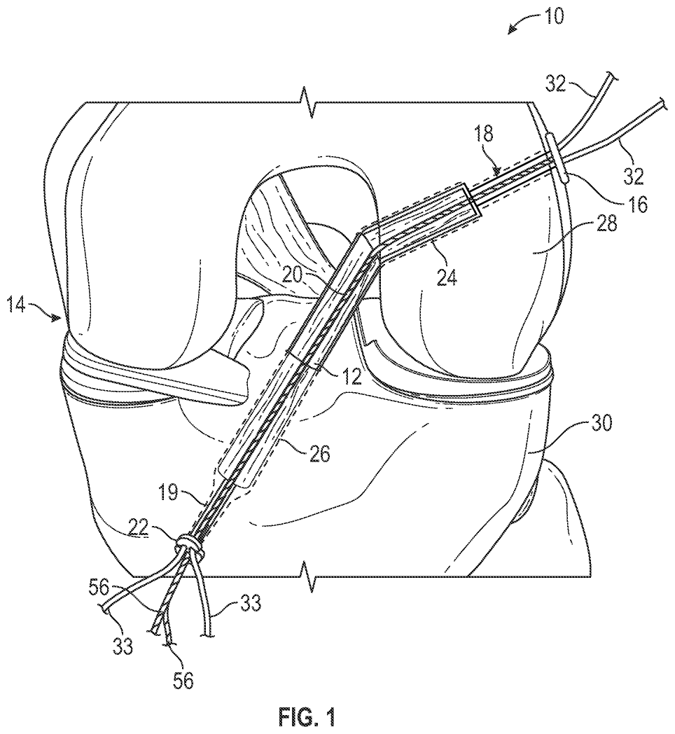

FIG. 1 illustrates an exemplary graft fixation system 10 for fixating a graft 12 within a joint 14 (e.g., a knee joint). The graft fixation system 10 may be used to fixate the graft 12 when performing a variety of tissue reconstruction procedures, for example. The tissue reconstruction procedures could include any surgical procedure in which it is desirable to position a replacement graft within a bone tunnel to repair torn tissue. ACL and PCL reconstructions are but two non-limiting examples of surgical reconstruction procedures that could benefit from the graft fixation systems 10 described herein. Other surgical procedures that could benefit from the teachings of this disclosure include but are not limited to acromioclavicular (AC) joint repairs, for example.

The graft fixation system 10 may be implanted within the joint 14 to repair a torn tissue (e.g., a torn ACL). Prior to implanting the graft fixation system 10 within the joint 14, a first bone tunnel 24 (e.g., a socket) may be formed in a first bone 28 (e.g., a femur), and a second bone tunnel 26 (e.g., a passage) may be formed in a second bone 30 (e.g., a tibia).

The graft fixation system 10 may include, among various other components or features, a cortical button 16, a first loop 18, a second loop 19, a reinforcement suture 20, and a surgical locking button 22. The cortical button 16 may be configured to provide suspensory cortical bone fixation of the graft 12 relative to the first bone 28 after the graft 12 has been positioned within the first bone tunnel 24. The cortical button 16 may include one or more apertures formed through the body of the cortical button 16 for receiving and carrying both the first loop 18 and the reinforcement suture 20. The cortical button 16 may be oblong or round and may be made of either metallic or polymeric materials within the scope of this disclosure.

The first loop 18 may be an adjustable loop made of a flexible material (e.g., suture) and includes an adjustable length and perimeter. Tensioning strands 32 of the first loop 18 may be pulled to reduce the size of the first loop 18. For example, the first loop 18 may be reduced in size by pulling the tensioning stands 32 in a first direction but is prevented from loosening in the opposite direction due to applied internal tensile forces. The tensioning strands 32 may be spliced through portions of the first loop 18 for establishing splices that are configured to constrict when tensioned to create the applied internal tensile forces.

The second loop 19 may also be an adjustable loop made of a flexible material (e.g., suture) and includes an adjustable length and perimeter. Tensioning strands 33 of the second loop 19 may be pulled to reduce the size of the second loop 19. For example, the second loop 19 may be reduced in size by pulling the tensioning stands 33 in a first direction but is prevented from loosening in the opposite direction due to applied internal tensile forces. The tensioning strands 33 may be spliced through portions of the second loop 19 for establishing splices that are configured to constrict when tensioned to create the applied internal tensile forces.

The first loop 18 may connect to the cortical button 16, and the second loop 19 may connect to the surgical locking button 22. The graft 12 may extend between the first loop 18 and the second loop 19 and may therefore be connected to the cortical button 16 and the surgical locking button 22 by the first loop 18 and the second loop 19, respectively. The graft 12 may be folded or looped over a cradle portion of each of the first loop 18 and the second loop 19. Alternatively, the graft 12 could connected to the surgical locking button 22 by a suture that is whipstitched to the graft 12, such as by tying a knot over top of the surgical locking button, for example.

The reinforcement suture 20 may be connected to the cortical button 16. For example, the reinforcement suture 20 may be received through apertures formed through the cortical button 16. The reinforcement suture 20 is therefore unconnected to the graft 12 and can be tensioned separately from the graft 12. For example, the graft 12 and the reinforcement suture 20 can be tensioned separately from one another, resulting in independent tension loads. Tension can be applied to the graft 12 (e.g., by tensioning the first loop 18 and/or the second loop 19) after tensioning the reinforcement suture 20. Accordingly, joint loads may be shared between the graft 12 and the reinforcement suture 20, with the reinforcement suture 20 acting as a dynamic joint stabilizer that shares loads with and reinforces the primary repair provided by the graft 12. The reinforcement suture 20 may therefore be referred to as an “internal brace” or “safety belt” feature of the graft fixation system 10.

The reinforcement suture 20 may include one or more strands of suture tape, such as FiberTape®, for example. FiberTape® is a suture product marketed and sold by Arthrex, Inc. However, other suture products could be utilized for the reinforcement suture 20 within the scope of this disclosure. The reinforcement suture 20 can include either a constant thickness or a varying thickness.

In an embodiment, the graft fixation system 10 may be implanted within the joint 14 by passing the cortical button 16 through the first bone tunnel 24. The cortical button 16 may be pulled through the first bone tunnel 24 using a passing suture (not shown) and can self-flip onto the cortex of the first bone 28 once tension is released on the passing suture. Alternatively, a flipping suture (not shown) could be used to flip and seat the cortical button 16 relative to the cortex of the first bone 28.

After seating the cortical button 16, the first loop 18 suspends the graft 12 within the first bone tunnel 24. The tensioning strands 32 of the first loop 18 can be pulled to adjust the size of the first loop 18 and to fully seat the graft 12 within the first bone tunnel 24.

The graft 12, the second loop 19, and the reinforcement suture 20 may subsequently be arranged to extend within the second bone tunnel 26. The tensioning strands 33 of the second loop 19 can be pulled to adjust the size of the second loop 19 and to fully seat the graft 12 within the second bone tunnel 26.

The graft 12 and the reinforcement suture 20 can be separately tensioned prior to completing the technique. For example, a first tension can be applied to the graft 12 by applying traction to the tensioning strands 33 of the second loop 19 (or to sutures that are whipstitched to the graft 12), and a second, different tension can be applied by applying traction to free ends 56 of the reinforcement suture 20 since these components are unconnected to one another. The graft 12 can thus be retensioned after intraoperative preconditioning. Intraoperative preconditioning can be used to reduce residual laxity in the graft 12. Graft retensioning optimizes the mechanical stability of soft tissue by decreasing its dynamic elongation. The reinforcement suture 20 may provide increased stiffness compared to soft tissue material, thus its resistance to dynamic elongation may be high enough without retensioning.

The surgical locking button can be configured to fixate both the graft 12 and the reinforcement suture 20 relative to the second bone 30. In prior systems and techniques, a separate screw, anchor or button was typically necessary to fixate the reinforcement suture 20 when securing the graft 12 relative to the second bone 30. However, as further discussed below, the surgical locking button 22 may be specifically configured to provide a common point of fixation for fixating both the graft 12 and the reinforcement suture 20 relative to the second bone 30.

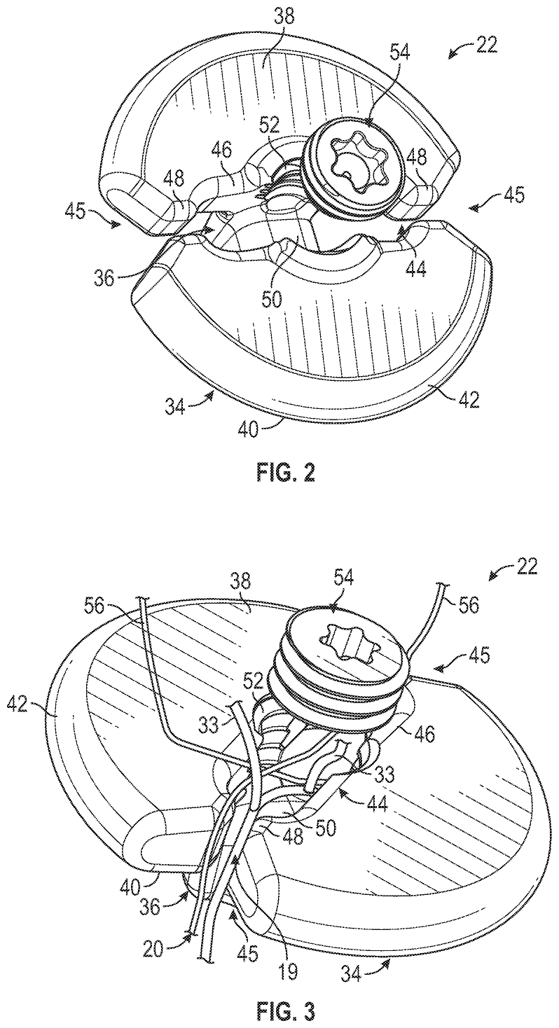

FIGS. 2 and 3, with continued reference to FIG. 1, illustrate an exemplary surgical locking button 22 that can be utilized as part of the graft fixation system 10 described above. The surgical locking button 22 may include a disk portion 34 and a collar portion 36 that protrudes from the disk portion 34. The disk portion 34 and the collar portion 36 may be formed together as part of a unitary structure.

The disk portion 34 of the surgical locking button 22 may include a top surface 38, a bottom surface 40, and a side wall 42 that extends between the top surface 38 and the bottom surface 40. At least a portion of the side wall 42 may include a circumferential surface for establishing the disk-like shape of the disk portion 34. One or more scallops 45 may be formed in the side wall 42.

The collar portion 36 may protrude outwardly from the bottom surface 40 of the disk portion 34. The surgical locking button 22 may be positioned relative to the second bone 30 such that the collar portion 36 is accommodated within the second bone tunnel 26 and the disk portion 34 is seated relative to the cortex of the second bone 30. In the seated position, the top surface 38 of the disk portion 34 faces away from the second bone 30 and the bottom surface 40 faces toward the second bone 30.

A slot 44 may bisect across a center of the disk portion 34. The slot 44 may be configured to accommodate one or more sutures, such as portions of the second loop 19, sutures that are whipstitched to the graft 12, and/or the free ends 56 of the reinforcement suture 20, within the surgical locking button 22.

The slot 44 may include a mid-section 46 that is located between a pair of longitudinal ends 48 that include a reduced width compared to the mid-section 46. Each of the longitudinal ends 48 of the slot 44 may open into one of the scallops 45 and may extend from the top surface 38 to the bottom surface 40. The mid-section 46 of the slot 44 may extend from the top surface 38 to a proximal surface of the collar portion 36. The proximal surface of the collar portion 36 establishes a bridge 50 of the surgical locking button 22. The bridge 50 can be used to connect the second loop 19 (or sutures that are whipstitched to the graft 12) and the reinforcement suture 20 to the surgical locking button 22. For example, the second loop 19 may be either looped or tied over the bridge 50.

A threaded opening 52 may be formed in the mid-section 46 of the slot 44. The threaded opening 52 may be provided at the center of the disk portion 34 and can be sized to receive a screw 54.

After arranging portions of the second loop 19 (or sutures that are whipstitched to the graft 12) and the reinforcement suture 20 within the slot 44 and over top of the bridge 50, the screw 54 can be inserted into the threaded opening 52 to clamp the second loop 19 (or sutures that are whipstitched to the graft 12) between a tip of the screw 54 and the bridge 50, thereby locking the sutures in place. The surgical locking button 22 is therefore equipped to provide the dual function of both fixating the graft 12 and the reinforcement suture 20 relative to the second bone 30.

The free ends 56 of the reinforcement suture 20 may be crisscrossed over one another over top of the bridge 50 and then tensioned prior to inserting the screw 54 into the threaded opening 52. The screw 54 prevents the reinforcement suture 20 from backing up or otherwise moving back into the second bone tunnel 26 one tightened down into the threaded opening 52. The tensioning strands 33 of the second loop 19 are generally not trapped under the screw 54 such that tension can be applied to the graft 12 even after fixation of the surgical locking button 22 at the cortex of the second bone 30.

Although shown as locking a single reinforcement suture 20 in the above implementations, the surgical locking button 22 could be configured to receive and lock multiple strands of suture via the screw 54.

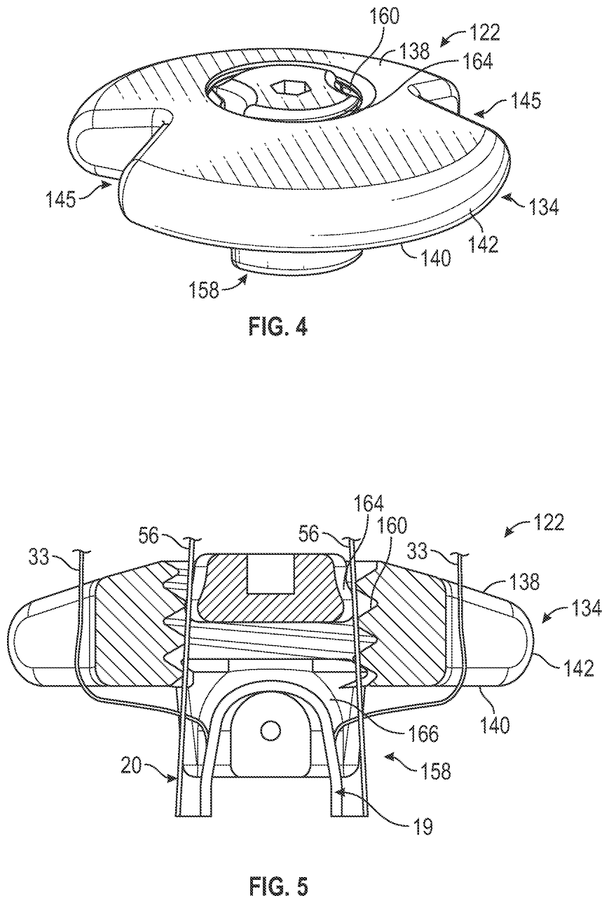

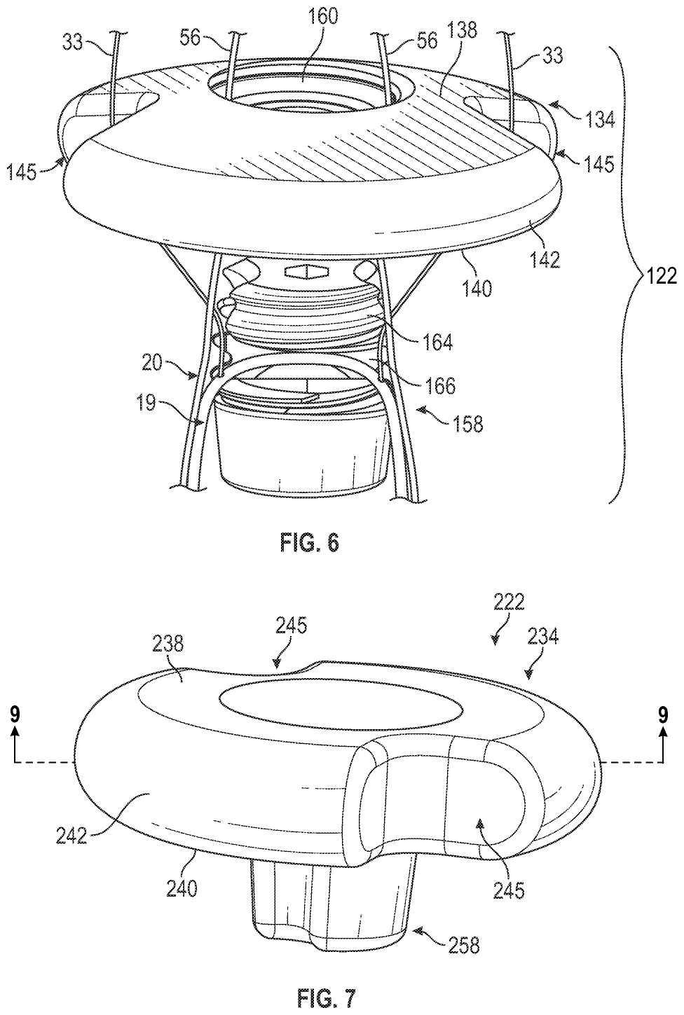

FIGS. 4, 5, and 6, with continued reference to FIG. 1, illustrate another exemplary surgical locking button 122 that can be utilized as part of the graft fixation system 10 described above. The surgical locking button 122 may include a disk portion 134 and an eyelet 158 that can be connected to the disk portion 134.

The disk portion 134 of the surgical locking button 122 may include a top surface 138, a bottom surface 140, and a side wall 142 that extends between the top surface 138 and the bottom surface 140. At least a portion of the side wall 142 may include a circumferential surface for establishing the disk-like shape of the disk portion 134. One or more scallops 145 may be formed in the side wall 142.

A threaded opening 160 may be formed through the disk portion 134. The threaded opening 160 may be provided at the center of the disk portion 134 and may extend completely through the disk portion 134. The threaded opening 160 may therefore open through both the top surface 138 and the bottom surface 140. The threaded opening 160 may be engaged by a threaded portion 164 of the eyelet 158 for removably securing (e.g., by screwing) the eyelet 158 to the disk portion 134.

When connected to the disk portion 134, the eyelet 158 may protrude outwardly away from the bottom surface 140 of the disk portion 134. The surgical locking button 122 may be positioned relative to the second bone 30 such that at least a portion of the eyelet 158 is accommodated within the second bone tunnel 26 and the disk portion 134 is seated relative to the cortex of the second bone 30. In the seated position, the top surface 138 of the disk portion 134 faces away from the second bone 30 and the bottom surface 140 faces toward the second bone 30.

A through-hole 166 may be formed through the eyelet 158 at a location that is distal to the threaded portion 164. The through-hole 166 may be configured to accommodate one or more sutures, such as portions of the second loop 19 or sutures that are whipstitched to the graft 12, for example. In an exemplary implementation, the second loop 19 may be accommodated within the through-hole 166 for connecting the graft 12 to the surgical locking button 122. The tensioning strands 33 of the second loop 19 may extend upwardly through the scallops 145 and can be tensioned for tensioning the graft 12 even after fixation of the surgical locking button 122 at the cortex of the second bone 30.

After passing free ends 56 of the reinforcement suture 20 through the threaded opening 160, the threaded portion 164 of the eyelet 158 can be screwed into the threaded opening 160 to trap the free ends 56 of the reinforcement suture 20 between the respective threads of the threaded opening 160 and the threaded portion 164, thereby locking the reinforcement suture 20 in place. The surgical locking button 122 is therefore equipped to provide the dual function of both fixating the graft 12 and the reinforcement suture 20 relative to the second bone 30.

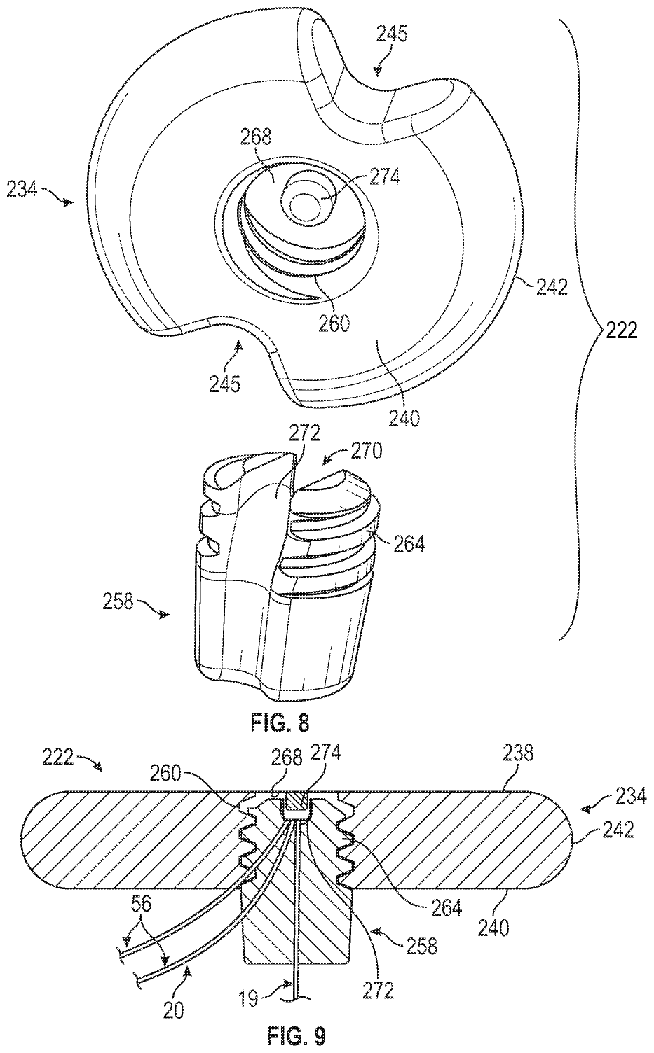

FIGS. 7, 8, and 9, with continued reference to FIG. 1, illustrate another exemplary surgical locking button 222 that can be utilized as part of the graft fixation system 10 described above. The surgical locking button 222 may include a disk portion 234 and an eyelet 258 that can be connected to the disk portion 234.

The disk portion 234 of the surgical locking button 222 may include a top surface 238, a bottom surface 240, and a side wall 242 that extends between the top surface 238 and the bottom surface 240. At least a portion of the side wall 242 may include a circumferential surface for establishing the disk-like shape of the disk portion 234. One or more scallops 245 may be formed in the side wall 242.

A threaded opening 260 may be formed in the bottom surface 240 of the disk portion 234. The threaded opening 260 may be provided at the center of the disk portion 234 and may extend from the bottom surface 240 to an interior wall 268 of the top surface 238. The threaded opening 260 may be engaged by a threaded portion 264 of the eyelet 258 for securing (e.g., by screwing) the eyelet 258 to the disk portion 234.

A pin 274 may protrude inwardly from the interior wall 268. The pin 274 therefore extends at least partially into the threaded opening 260. The pin 274 may be centered relative to a longitudinal centerline of the threaded opening 260.

When connected to the disk portion 234, the eyelet 258 may protrude outwardly away from the bottom surface 240. The surgical locking button 222 may be positioned relative to the second bone 30 such that at least a portion of the eyelet 258 is accommodated within the second bone tunnel 26 and the disk portion 234 is seated relative to the cortex of the second bone 30. In the seated position, the top surface 238 of the disk portion 234 faces away from the second bone 30 and the bottom surface 240 faces toward the second bone 30.

A slot 270 may bisect the threaded portion 264 of the eyelet 258 into two halves. A bridge 272 may establish a base of the slot 270. The bridge 272 can be used to connect the second loop 19 (or sutures that are whipstitched to the graft 12) and the reinforcement suture 20 to the surgical locking button 222.

After arranging portions of the second loop 19 (or sutures that are whipstitched to the graft 12) and the reinforcement suture 20 within the slot 270 and over top of the bridge 272, the threaded portion 264 of the eyelet 258 can be screwed into the threaded opening 260 to clamp the various strands of sutures between a tip of the pin 274 and the bridge 272, thereby locking the sutures in place. The surgical locking button 222 is therefore equipped to provide the dual function of both fixating the graft 12 and the reinforcement suture 20 relative to the second bone 30.

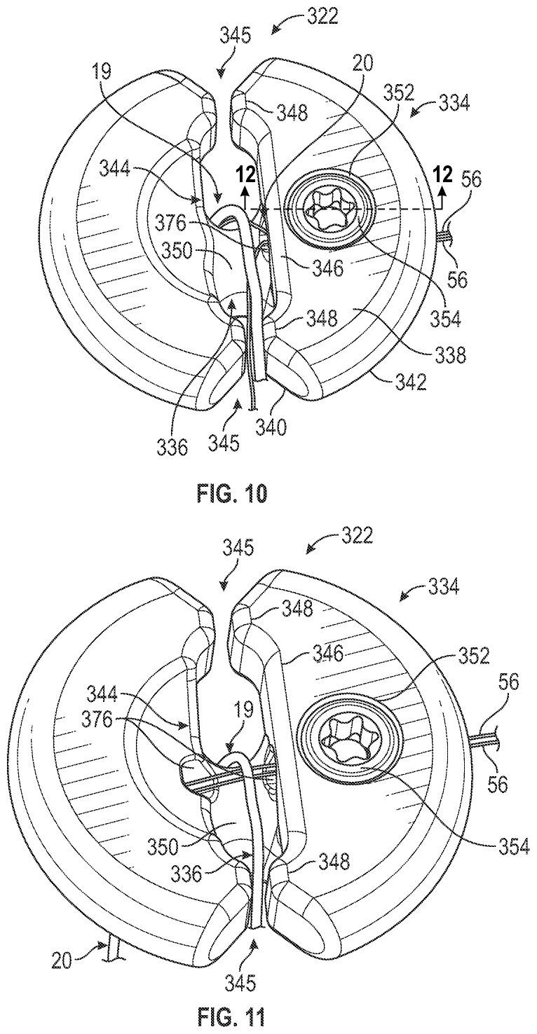

FIGS. 10, 11, and 12 illustrate another exemplary surgical locking button 322 that can be utilized as part of the graft fixation system 10 described above. The surgical locking button 322 may include a disk portion 334 and a collar portion 336 that protrudes from the disk portion 334. The disk portion 334 and the collar portion 336 may be formed together as part of a unitary structure.

The disk portion 334 of the surgical locking button 322 may include a top surface 338, a bottom surface 340, and a side wall 342 that extends between the top surface 338 and the bottom surface 340. At least a portion of the side wall 342 may include a circumferential surface for establishing the disk-like shape of the disk portion 334. One or more scallops 345 may be formed in the side wall 342.

The collar portion 336 may protrude outwardly from the bottom surface 340 of the disk portion 334. The surgical locking button 322 may be positioned relative to the second bone 30 such that the collar portion 336 is accommodated within the second bone tunnel 26 and the disk portion 334 is seated relative to the cortex of the second bone 30. In the seated position, the top surface 338 of the disk portion 334 faces away from the second bone 30 and the bottom surface 340 faces toward the second bone 30.

A slot 344 may bisect across a center of the disk portion 334. The slot 344 may be configured to accommodate one or more sutures, such as portions of the second loop 19, sutures that are whipstitched to the graft 12, and/or the reinforcement suture 20, within the surgical locking button 322.

The slot 344 may include a mid-section 346 that is located between a pair of longitudinal ends 348. Each of the longitudinal ends 348 of the slot 344 may open into one of the scallops 345 and may extend from the top surface 338 to the bottom surface 340. The mid-section 346 of the slot 344 may extend from the top surface 338 to a proximal surface of the collar portion 336. The proximal surface of the collar portion 336 establishes a bridge 350 of the surgical locking button 322. The bridge 350 can be used to connect the second loop 19 (or sutures that are whipstitched to the graft 12) and the reinforcement suture 20 to the surgical locking button 322.

A threaded opening 352 may be formed in the disk portion 334 at a location that is offset from the slot 344. The threaded opening 352 may therefore be provided at an off-center location of the disk portion 334 and can be sized to receive a screw 354. The threaded opening 352 may be formed through the top surface 338 but terminates prior to piercing through the bottom surface 340.

A suture tunnel 376 may also be formed in the disk portion 334. The suture tunnel 376 may extend inside the disk portion 334 at a location beneath the threaded opening 352 and may include a first end that opens at the bridge 350 and a second end that opens through the side wall 342 of the disk portion 334. The suture tunnel 376 therefore extends along a longitudinal axis that is transverse to longitudinal axis of the slot 344. The threaded opening 352 may open into the suture tunnel 376.

The surgical locking button 322 of FIG. 10 includes one suture tunnel 376 for accommodating passage of the reinforcement suture 20. However, the surgical locking button 322 could include additional suture tunnels 376 within the scope of this disclosure (see, e.g., the implementation shown in FIG. 11).

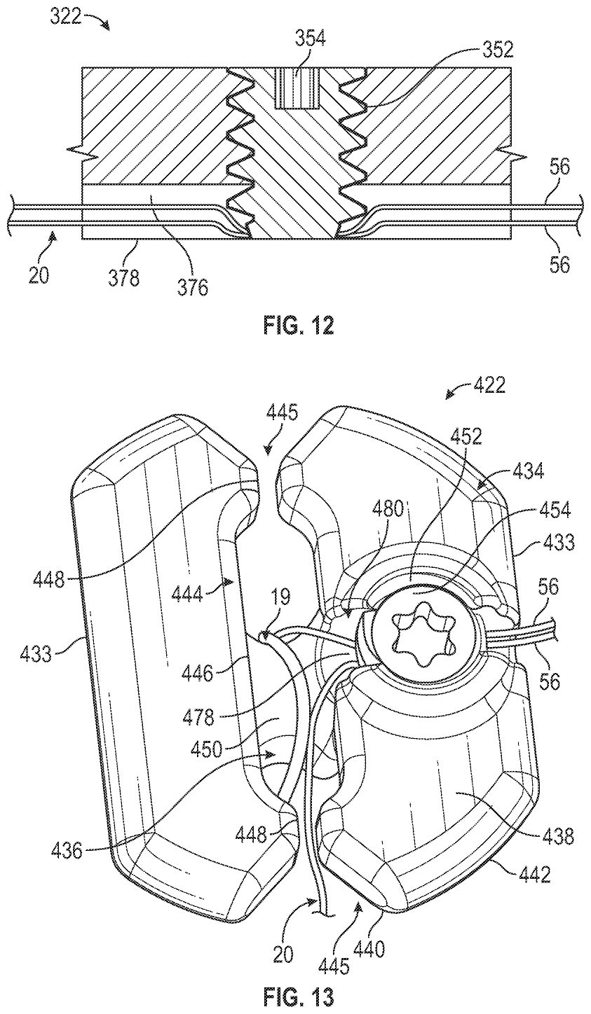

After passing free ends 56 of the reinforcement suture 20 through the one or more suture tunnels 376, the screw 354 can be inserted into the threaded opening 352 to clamp the reinforcement suture 20 between a tip of the screw 354 and a floor 378 (see FIG. 12) of the suture tunnel 376, thereby locking the reinforcement suture 20 in place. The surgical locking button 222 is therefore equipped to provide the dual function of both fixating the graft 12 and the reinforcement suture 20 relative to the second bone 30.

FIG. 13 illustrates another exemplary surgical locking button 422 that can be utilized as part of the graft fixation system 10 described above. The surgical locking button 422 may include a disk portion 434 and a collar portion 436 that protrudes from the disk portion 434. The disk portion 434 and the collar portion 436 may be formed together as part of a unitary structure.

The disk portion 434 of the surgical locking button 422 may include a top surface 438, a bottom surface 440, and a side wall 442 that extends between the top surface 438 and the bottom surface 440. At least a portion of the side wall 442 may include a circumferential surface for establishing the disk-like shape of the disk portion 434. However, the side wall 442 may also include one or more flat surfaces 433. One or more scallops 445 may be formed in the side wall 442.

The collar portion 436 may protrude outwardly from the bottom surface 440 of the disk portion 434. The surgical locking button 422 may be positioned relative to the second bone 30 such that the collar portion 436 is accommodated within the second bone tunnel 26 and the disk portion 434 is seated relative to the cortex of the second bone 30. In the seated position, the top surface 438 of the disk portion 434 faces away from the second bone 30 and the bottom surface 440 faces toward the second bone 30.

A first slot 444 may bisect across a center of the disk portion 434. The first slot 444 may be configured to accommodate one or more sutures, such as portions of the second loop 19, sutures that are whipstitched to the graft 12, and/or the reinforcement suture 20, within the surgical locking button 422.

The first slot 444 may include a mid-section 446 that is located between a pair of longitudinal ends 448. Each of the longitudinal ends 448 of the first slot 444 may open into one of the scallops 445 and may extend from the top surface 438 to the bottom surface 440. The mid-section 446 of the first slot 444 may extend from the top surface 438 to a proximal surface of the collar portion 436. The proximal surface of the collar portion 436 establishes a bridge 450 of the surgical locking button 422. The bridge 450 can be used to connect the second loop 19 (or sutures that are whipstitched to the graft 12) to the surgical locking button 422.

A second slot 480 may also be formed in the disk portion 434. The second slot 480 may include a first end that opens at the bridge 450 and a second end that opens through the side wall 442 of the disk portion 434. The second slot 480 therefore extends along a longitudinal axis that is transverse to the longitudinal axis of the first slot 444.

A threaded opening 452 may be formed in the disk portion 434 at a location that is offset from the first slot 444. The threaded opening 352 may be formed in the second slot 444. The threaded opening 452 may therefore be provided at an off-center location of the disk portion 434 and can be sized to receive a screw 454. The threaded opening 452 may be formed completely through the disk portion 434 and therefore opens through both the top surface 438 and the bottom surface 440.

After passing free ends 56 of the reinforcement suture 20 through the second slot 480, the screw 454 can be inserted into the threaded opening 452 to clamp the reinforcement suture 20 between a tip of the screw 454 and a floor 478 of the second slot 480, thereby locking the reinforcement suture 20 in place. The surgical locking button 422 is therefore equipped to provide the dual function of both fixating the graft 12 and the reinforcement suture 20 relative to the second bone 30.

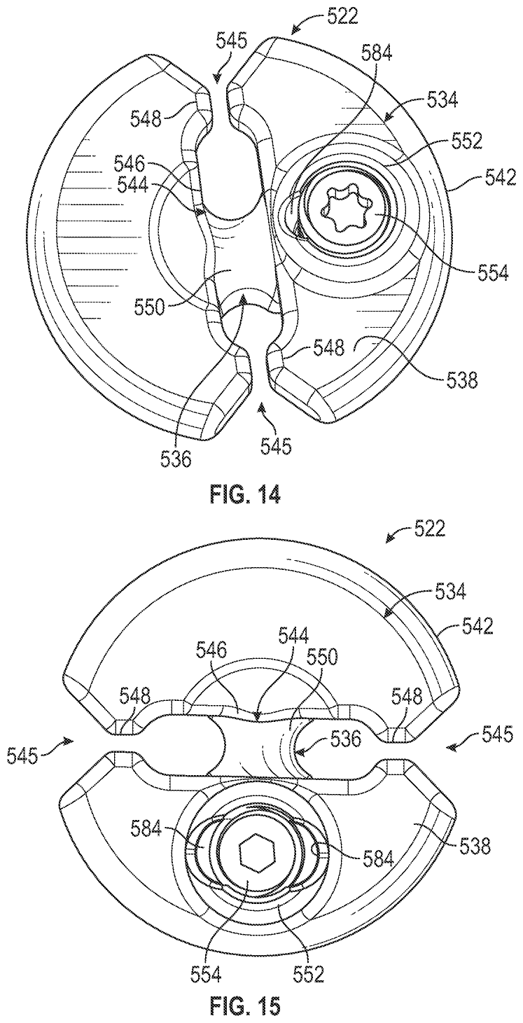

FIGS. 14, 15, and 16 illustrate another exemplary surgical locking button 522 that can be utilized as part of the graft fixation system 10 described above. The surgical locking button 522 may include a disk portion 534 and a collar portion 536 that protrudes from the disk portion 534. The disk portion 534 and the collar portion 536 may be formed together as part of a unitary structure.

The disk portion 534 of the surgical locking button 522 may include a top surface 538, a bottom surface 540, and a side wall 542 that extends between the top surface 538 and the bottom surface 540. At least a portion of the side wall 542 may include a circumferential surface for establishing the disk-like shape of the disk portion 534. One or more scallops 545 may be formed in the side wall 542.

The collar portion 536 may protrude outwardly away from the bottom surface 540 of the disk portion 534. The surgical locking button 522 may be positioned relative to the second bone 30 such that the collar portion 536 is accommodated within the second bone tunnel 26 and the disk portion 534 is seated relative to the cortex of the second bone 30. In the seated position, the top surface 538 of the disk portion 534 faces away from the second bone 30 and the bottom surface 540 faces toward the second bone 30.

A slot 544 may bisect across a center of the disk portion 534. The slot 544 may be configured to accommodate one or more sutures, such as portions of the second loop 19, sutures that are whipstitched to the graft 12, and/or the reinforcement suture 20, within the surgical locking button 522.

The slot 544 may include a mid-section 546 that is located between a pair of longitudinal ends 548. Each of the longitudinal ends 548 of the slot 544 may open into one of the scallops 545 and may extend from the top surface 538 to the bottom surface 540. The mid-section 546 of the slot 544 may extend from the top surface 538 to a proximal surface of the collar portion 536. The proximal surface of the collar portion 536 establishes a bridge 550 of the surgical locking button 522. The bridge 550 can be used to connect the second loop 19 (or sutures that are whipstitched to the graft 12) to the surgical locking button 522.

A threaded opening 552 may be formed in the disk portion 534 at a location that is offset from the slot 544. The threaded opening 552 may therefore be provided at an off-center location of the disk portion 534 and can be sized to receive a screw 554. One or more suture tunnels 584 may be formed in the threaded opening 552 for accommodating the passage of the reinforcement suture 20 through the threaded opening 552.

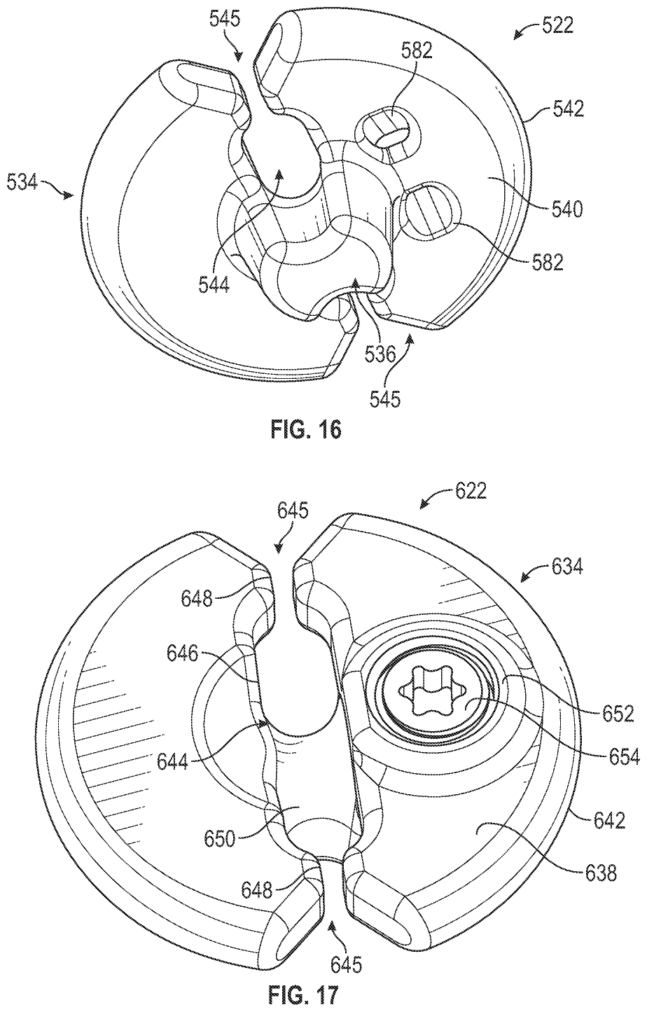

The threaded opening 552 may be formed through the disk portion 534 and therefore opens through both top surface 538 and the bottom surface 540. Alternatively, one or more suture holes 582 that open into the threaded opening 552 may be formed in the bottom surface 538 (see FIG. 16) for accommodating the passage of the reinforcement suture 20 through the surgical locking button 522.

After passing free ends 56 of the reinforcement suture 20 through the threaded opening 552 (e.g., in a direction that extends from the bottom surface 540 toward the top surface 538), the screw 554 can be inserted into the threaded opening 552 to trap the reinforcement suture between the threading of the screw 554 and the threading of the threaded opening 552, thereby locking the reinforcement suture 20 in place. The surgical locking button 522 is therefore equipped to provide the dual function of both fixating the graft 12 and the reinforcement suture 20 relative to the second bone 30.

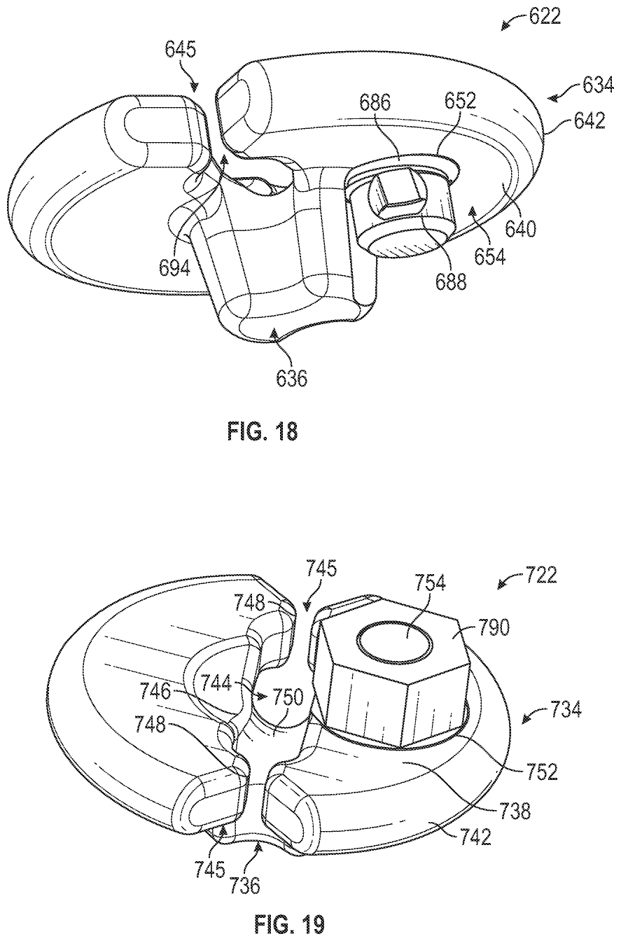

FIGS. 17 and 18 illustrate another exemplary surgical locking button 622 that can be utilized as part of the graft fixation system 10 described above. The surgical locking button 622 may include a disk portion 634 and a collar portion 636 that protrudes from the disk portion 634. The disk portion 634 and the collar portion 636 may be formed together as part of a unitary structure.

The disk portion 634 of the surgical locking button 622 may include a top surface 638, a bottom surface 640, and a side wall 642 that extends between the top surface 638 and the bottom surface 640. At least a portion of the side wall 642 may include a circumferential surface for establishing the disk-like shape of the disk portion 634. One or more scallops 645 may be formed in the side wall 642.

The collar portion 636 may protrude outwardly away from the bottom surface 640 of the disk portion 634. The surgical locking button 622 may be positioned relative to the second bone 30 such that the collar portion 636 is accommodated within the second bone tunnel 26 and the disk portion 634 is seated relative to the cortex of the second bone 30. In the seated position, the top surface 638 of the disk portion 634 faces away from the second bone 30 and the bottom surface 640 faces toward the second bone 30.

A slot 644 may bisect across a center of the disk portion 634. The slot 644 may be configured to accommodate one or more sutures, such as portions of the second loop 19 or sutures that are whipstitched to the graft 12, within the surgical locking button 622.

The slot 644 may include a mid-section 646 that is located between a pair of longitudinal ends 648. Each of the longitudinal ends 648 of the slot 644 may open into one of the scallops 645 and may extend from the top surface 638 to the bottom surface 640. The mid-section 646 of the slot 644 may extend from the top surface 638 to a proximal surface of the collar portion 636. The proximal surface of the collar portion 636 establishes a bridge 650 of the surgical locking button 622. The bridge 650 can be used to connect the second loop 19 (or sutures that are whipstitched to the graft 12) to the surgical locking button 622.

A threaded opening 652 may be formed in the disk portion 634 at a location that is offset from the slot 644. The threaded opening 652 may therefore be provided at an off-center location of the disk portion 634 and can be sized to receive a screw 654. The threaded opening 652 may be formed through the disk portion 634 and therefore opens through both top surface 638 and the bottom surface 640.

The screw 654 may include a threaded portion 686 and an eyelet 688. The threaded portion 686 may engage the threaded opening 652 for securing (e.g., by screwing) the screw 654 to the disk portion 634, and the eyelet 688 may be configured to receive free ends 56 of the reinforcement suture 20.

After passing the free ends 56 of the reinforcement suture 20 through the eyelet 688 of the screw 654, the threaded portion 686 of the screw 654 can be screwed into the threaded opening 652 to trap the free ends 56 of the reinforcement suture 20 between the screw 654 and the threaded opening 652, thereby locking the reinforcement suture 20 in place. The surgical locking button 622 is therefore equipped to provide the dual function of both fixating the graft 12 and the reinforcement suture 20 relative to the second bone 30

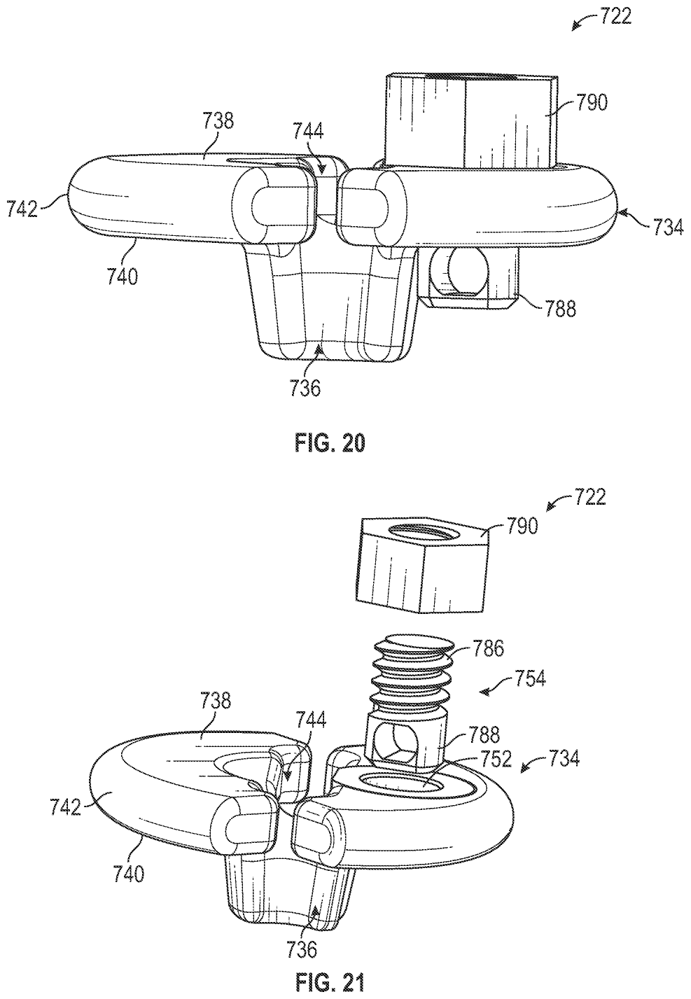

FIGS. 19, 20, and 21 illustrate another exemplary surgical locking button 722 that can be utilized as part of the graft fixation system 10 described above. The surgical locking button 722 may include a disk portion 734 and a collar portion 736 that protrudes from the disk portion 734. The disk portion 734 and the collar portion 736 may be formed together as part of a unitary structure.

The disk portion 734 of the surgical locking button 722 may include a top surface 738, a bottom surface 740, and a side wall 742 that extends between the top surface 738 and the bottom surface 740. At least a portion of the side wall 742 may include a circumferential surface for establishing the disk-like shape of the disk portion 734. One or more scallops 745 may be formed in the side wall 742.

The collar portion 736 may protrude away from the bottom surface 740 of the disk portion 734. The surgical locking button 722 may be positioned relative to the second bone 30 such that the collar portion 736 is accommodated within the second bone tunnel 26 and the disk portion 734 is seated relative to the cortex of the second bone 30. In the seated position, the top surface 738 of the disk portion 734 faces away from the second bone 30 and the bottom surface 740 faces toward the second bone 30.

A slot 744 may bisect across a center of the disk portion 734. The slot 744 may be configured to accommodate one or more sutures, such as portions of the second loop 19 or sutures that are whipstitched to the graft 12, within the surgical locking button 722.

The slot 744 may include a mid-section 746 that is located between a pair of longitudinal ends 748. Each of the longitudinal ends 748 of the slot 744 may open into one of the scallops 745 and may extend from the top surface 738 to the bottom surface 740. The mid-section 746 of the slot 744 may extend from the top surface 738 to a proximal surface of the collar portion 736. The proximal surface of the collar portion 736 establishes a bridge 750 of the surgical locking button 722. The bridge 750 can be used to connect the second loop 19 (or sutures that are whipstitched to the graft 12) to the surgical locking button 722.

An unthreaded opening 752 may be formed in the disk portion 734 at a location that is offset from the slot 744. The unthreaded opening 752 may therefore be provided at an off-center location of the disk portion 734 and can be sized to receive a screw 754. The unthreaded opening 752 may be formed through the disk portion 734 and therefore opens through both top surface 738 and the bottom surface 740.

The screw 754 may include a threaded portion 786 and an eyelet 788. The threaded portion 786 may engage an internal threading of a screw nut 790 for securing the screw 754 relative to the disk portion 734, and the eyelet 788 may be configured to receive free ends 56 of the reinforcement suture 20.

After passing the free ends 56 of the reinforcement suture 20 through the eyelet 788 of the screw 754 and positioning the threaded portion 786 through the unthreaded opening 752 of the disk portion 734, the screw nut 790 may be screwed onto the threaded portion 686 to pull the eyelet 788 into the disk portion 734 and trap the free ends 56 of the reinforcement suture 20 between the screw 754 and the unthreaded opening 752, thereby locking the reinforcement suture 20 in place. The surgical locking button 722 is therefore equipped to provide the dual function of both fixating the graft 12 and the reinforcement suture 20 relative to the second bone 30.

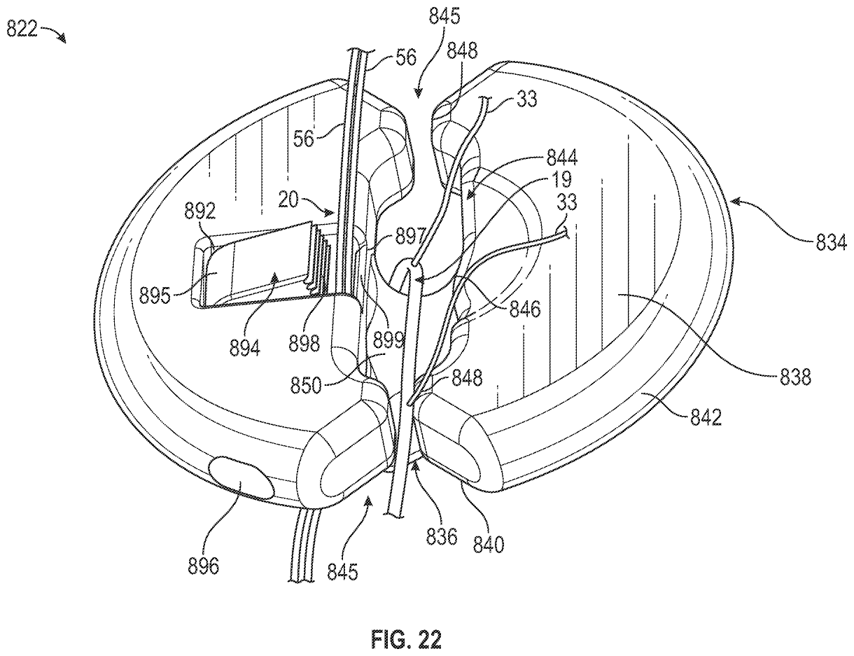

FIG. 22 illustrates another exemplary surgical locking button 822 that can be utilized as part of the graft fixation system 10 described above. The surgical locking button 822 may include a disk portion 834 and a collar portion 836 that protrudes from the disk portion 834. The disk portion 834 and the collar portion 836 may be formed together as part of a unitary structure.

The disk portion 834 of the surgical locking button 822 may include a top surface 838, a bottom surface 840, and a side wall 842 that extends between the top surface 838 and the bottom surface 840. At least a portion of the side wall 842 may include a circumferential surface for establishing the disk-like shape of the disk portion 834. One or more scallops 845 may be formed in the side wall 842.

The collar portion 836 may protrude outwardly away from the bottom surface 840 of the disk portion 834. The surgical locking button 822 may be positioned relative to the second bone 30 such that the collar portion 836 is at least partially accommodated within the second bone tunnel 26 and the disk portion 834 is seated relative to the cortex of the second bone 30. In the seated position, the top surface 838 of the disk portion 834 faces away from the second bone 30 and the bottom surface 840 faces toward the second bone 30.

A slot 844 may bisect across a center of the disk portion 834. The slot 844 may be configured to accommodate one or more sutures, such as portions of the second loop 19 or sutures that are whipstitched to the graft 12, within the surgical locking button 822.

The slot 844 may include a mid-section 846 that is located between a pair of longitudinal ends 848. Each of the longitudinal ends 848 of the slot 844 may open into one of the scallops 845 and may extend from the top surface 838 to the bottom surface 840. The mid-section 846 of the slot 844 may extend from the top surface 838 to a proximal surface of the collar portion 836. The proximal surface of the collar portion 836 establishes a bridge 850 of the surgical locking button 822. The bridge 850 can be used to connect the second loop 19 (or sutures that are whipstitched to the graft 12) to the surgical locking button 822.

A through passage 892 may be formed through the disk portion 834 and therefore extends through both the top surface 838 and the bottom surface 840. The through passage 892 may be formed at a location that is offset from the slot 844 and is therefore located at an off-center location of the disk portion 834. The through passage 892 may extend along a longitudinal axis that is transverse to longitudinal axis of the slot 844.

A door 894 may be secured within the through passage 892 by a cross-pin 896. The door 894 can pivot about the cross-pin 896 in order to move between an open position and a closed position relative to the disk portion 834.

The door 894 may include a plurality of teeth 898 that can engage a plurality of teeth 899 of a wall 897 of the disk portion 834 when the door 894 is pivoted to the closed position. The wall 897 establishes a portion of the perimeter of the through passage 892 and can be located immediately adjacent to the bridge 850. The teeth 898 and the teeth 899 are spaced from one another when the door 894 is in the open position. A user can manually move the door 894 between the closed position and the open position by applying thumb pressure near an end portion 895 of the door 894 that is located on an opposite end of the door 894 from the teeth 898.

The free ends 56 of the reinforcement suture 20 may be passed through the through passage 892 when the door 894 is moved to the open position. After passing the free ends 56 of the reinforcement suture 20 through the through passage 892, the door 894 may be pivoted to the closed position to trap the free ends 56 of the reinforcement suture 20 between the teeth 898, 899, thereby locking the reinforcement suture 20 in place. The surgical locking button 822 is therefore equipped to provide the dual function of both fixating the graft 12 and the reinforcement suture 20 relative to the second bone 30.

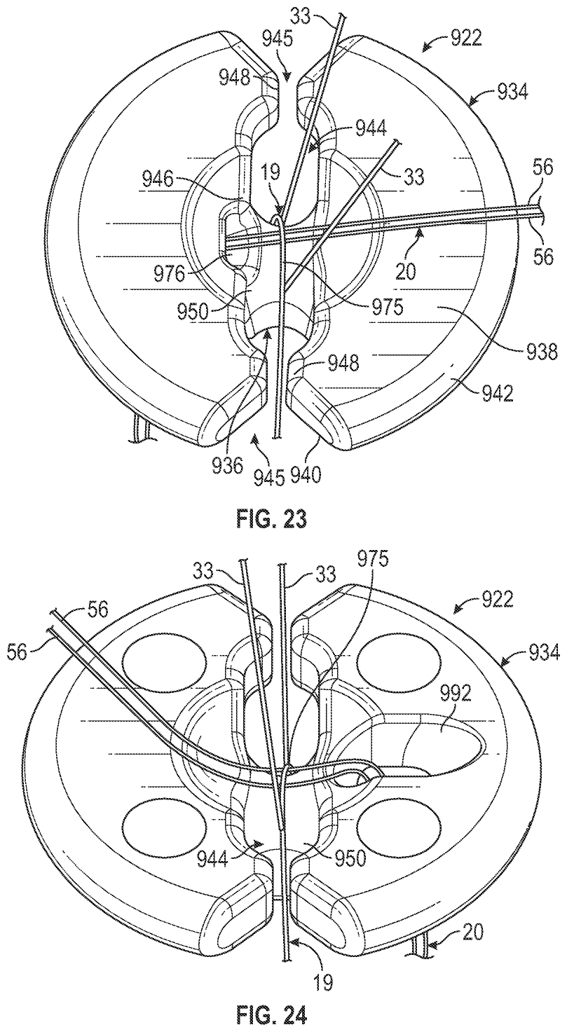

FIGS. 23, 24, and 25 illustrate yet another exemplary surgical locking button 922 that can be utilized as part of the graft fixation system 10 described above. The surgical locking button 922 may include a disk portion 934 and a collar portion 936 that protrudes from the disk portion 934. The disk portion 934 and the collar portion 936 may be formed together as part of a unitary structure.

The disk portion 934 of the surgical locking button 922 may include a top surface 938, a bottom surface 940, and a side wall 942 that extends between the top surface 938 and the bottom surface 940. At least a portion of the side wall 942 may include a circumferential surface for establishing the disk-like shape of the disk portion 934. One or more scallops 945 may be formed in the side wall 942.

The collar portion 936 may protrude outwardly from the bottom surface 940 of the disk portion 934. The surgical locking button 922 may be positioned relative to the second bone 30 such that the collar portion 936 is accommodated within the second bone tunnel 26 and the disk portion 934 is seated relative to the cortex of the second bone 30. In the seated position, the top surface 938 of the disk portion 934 faces away from the second bone 30 and the bottom surface 940 faces toward the second bone 30.

A slot 944 may bisect across a center of the disk portion 934. The slot 944 may be configured to accommodate one or more sutures, such as portions of the second loop 19 or sutures that are whipstitched to the graft 12, within the surgical locking button 922.

The slot 944 may include a mid-section 946 that is located between a pair of longitudinal ends 948. Each of the longitudinal ends 948 of the slot 944 may open into one of the scallops 945 and may extend from the top surface 938 to the bottom surface 940. The mid-section 946 of the slot 944 may extend from the top surface 938 to a proximal surface of the collar portion 936. The proximal surface of the collar portion 936 establishes a bridge 950 of the surgical locking button 922. The bridge 950 can be used to connect the second loop 19 (or sutures that are whipstitched to the graft 12) and the reinforcement suture 20 to the surgical locking button 922.

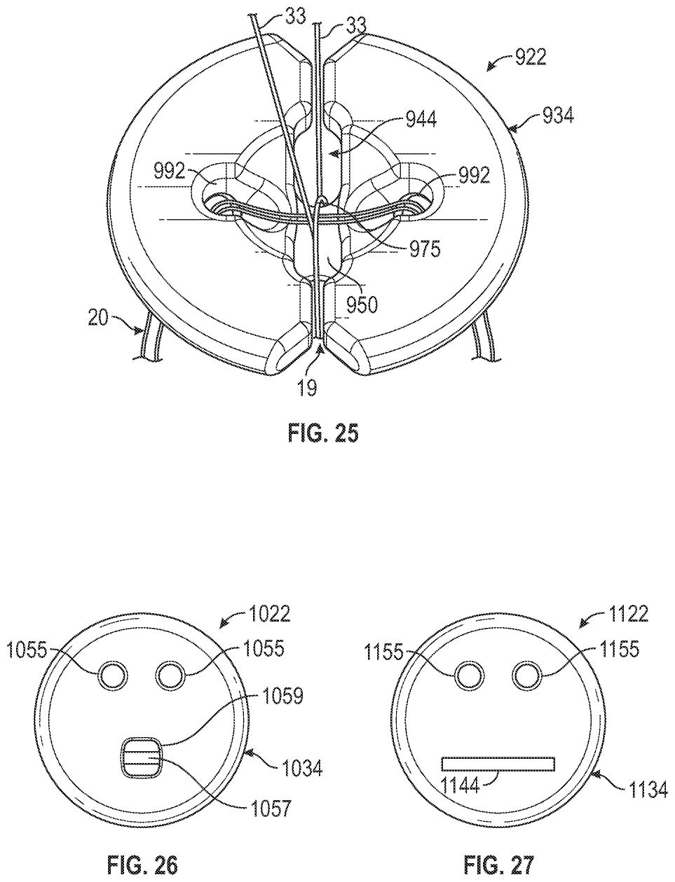

One or more suture tunnels 976 (see FIG. 23) or one or more through passages 992 (see FIGS. 24 and 25) may be formed in the disk portion 934. The suture tunnel 976 may may include a first end that opens at the bridge 950 and a second end that opens through the side wall 942 of the disk portion 934. The suture tunnel 976 can therefore extend along a longitudinal axis that is transverse to longitudinal axis of the slot 944. The through passage 992 may be formed through the disk portion 934 and therefore extends through both the top surface 938 and the bottom surface 940. The through passage 992 may be formed at a location that is offset from the slot 944 and is therefore located at an off-center location of the disk portion 934.

After passing the free ends 56 of the reinforcement suture 20 through either the suture tunnel 976 or the through passage 992, the free ends 56 may be passed beneath a loop section 975 of the second loop 19. The second loop 19 may be tensioned (e.g., by applying traction to the tensioning stands 33) to trap the reinforcement suture 20 between the loop section 975 and the bridge 950, thereby locking the reinforcement suture 20 in place. The surgical locking button 922 is therefore equipped to provide the dual function of both fixating the graft 12 and the reinforcement suture 20 relative to the second bone 30.

FIG. 26 illustrates yet another exemplary surgical locking button 1022 that can be utilized as part of the graft fixation system 10 described above. The surgical locking button 1022 may include a body 1034 having a disk-like shape. However, other shapes are contemplated within the scope of this disclosure.

A pair of apertures 1055 may be formed through the body 1034 and may extend through both a top surface and the bottom surface of the body 1034. The apertures 1055 can be used to connect the second loop 19 (or sutures that are whipstitched to the graft 12) to the surgical locking button 1022.

A movable pin 1057 may be mounted within an additional opening 1059 that is formed through the body 1034. The reinforcement suture 20 can be routed through the opening 1059 and around the movable pin 1057 for locking the reinforcement suture 20 in place.

FIG. 27 illustrates yet another exemplary surgical locking button 1122 that can be utilized as part of the graft fixation system 10 described above. The surgical locking button 1122 may include a body 1134 having a disk-like shape. However, other shapes are contemplated within the scope of this disclosure.

A pair of apertures 1155 may be formed through the body 1134 and may extend through both a top surface and the bottom surface of the body 1134. The apertures 1155 can be used to connect the second loop 19 (or sutures that are whipstitched to the graft 12) to the surgical locking button 1122.

A slot 1144 may also be formed through the body 1134. The reinforcement suture 20 may be secured within the slot 1144 via a friction fit. For example, the reinforcement suture 20 may include a varying thickness and thus includes one or more tapered regions where the reinforcement suture 20 transitions between a thickened section and a thinned section. Thinned sections of the reinforcement suture 20 may be pulsed through the slot 114 until the thickened sections wedge into the slot 1144 the lock the reinforcement suture 20 in place.

The graft fixation systems and surgical methods described herein may be utilized to secure grafts within bone tunnels. The graft fixation systems may include a surgical locking button that includes features for fixating both a suture loop and a reinforcement suture that is separate from an unattached to the suture loop or a graft that is carried by the suture loop. Accordingly, a single fixation device can be utilized for achieving fixation of both the graft and the reinforcement suture.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A surgical locking button, comprising:
a disk portion;
a collar portion protruding from the disk portion;
a proximal surface of the collar portion establishing a bridge for securing a graft relative to a bone; and
a screw or a door connectable to the disk portion and configured for locking a reinforcement suture relative to the bone.

2. The surgical locking button as recited in claim 1, wherein the disk portion and the collar portion establish a unitary body of the surgical locking button.

3. The surgical locking button as recited in claim 1, comprising a slot that bisects the disk portion.

4. The surgical locking button as recited in claim 3, comprising a threaded opening formed in the slot and configured to receive the screw for trapping the reinforcement suture between the screw and the bridge.

5. The surgical locking button as recited in claim 3, comprising a threaded opening formed in the disk portion at a location that is offset from the slot, wherein the threaded opening is configured to receive the screw for trapping the reinforcement suture between the screw and a floor of a suture tunnel that extends beneath the threaded opening.

6. The surgical locking button as recited in claim 3, comprising a threaded opening formed in the disk portion at a location that is offset from the slot, wherein the threaded opening is configured to receive the screw for trapping the reinforcement suture between the screw and a floor of a second slot that bisects the threaded opening.

7. The surgical locking button as recited in claim 3, comprising a threaded opening formed in the disk portion at a location that is offset from the slot, wherein the threaded opening is configured to receive the screw for trapping the reinforcement suture between a first threading of the screw and a second threading of the threaded opening.

8. The surgical locking button as recited in claim 3, comprising a threaded opening formed in the disk portion at a location that is offset from the slot, wherein the threaded opening is configured to receive the screw for trapping the reinforcement suture between an eyelet of the screw and the threaded opening.

9. The surgical locking button as recited in claim 3, comprising an unthreaded opening formed in the disk portion at a location that is offset from the slot, wherein the unthreaded opening is configured to receive the screw for trapping the reinforcement suture between an eyelet of the screw and the unthreaded opening.

10. The surgical locking button as recited in claim 9, wherein the screw is secured within the unthreaded opening by a screw nut.

11. The surgical locking button as recited in claim 3, comprising a through passage formed in the disk portion at a location that is offset from the slot, wherein the door is pivotably mounted within the through passage by a cross-pin.

12. The surgical locking button as recited in claim 11, wherein the door includes a first set of teeth configured to engage a second set of teeth of the disk portion for locking the reinforcement suture relative to the disk portion.

13. A surgical locking button, comprising:
a disk portion including a threaded opening; and
an eyelet connectable to the disk portion and including a threaded portion that is configured to engage the threaded opening,
wherein the eyelet is configured to secure both a graft and a reinforcement suture relative to a bone.

14. The surgical locking button as recited in claim 13, wherein a through-hole of the eyelet is configured to receive a suture loop for securing the graft relative to the bone, and further wherein the threaded opening is configured to receive the threaded portion for trapping the reinforcement suture between a first threading of the eyelet and a second threading of the threaded opening.

15. The surgical locking button as recited in claim 13, wherein a suture loop connected to the graft and the reinforcement suture are clamped between a pin of the disk portion and a bridge of the eyelet when the eyelet is connected to the disk portion.

16. A surgical method, comprising:
positioning a surgical locking button relative to a bone tunnel of a bone,
wherein the surgical locking button fixates both a graft and a reinforcement suture relative to the bone.

17. The surgical method as recited in claim 16, wherein the graft is secured to the surgical locking button by a suture or a suture loop.

18. The surgical method as recited in claim 16, wherein a screw or a door of the surgical locking button locks a position of the reinforcement suture relative to the surgical locking button.

19. The surgical method as recited in claim 16, wherein an eyelet of the surgical locking button locks a position of the reinforcement suture relative to the surgical locking button.

20. The surgical method as recited in claim 16, wherein a suture loop that connects the graft to the surgical locking button locks a position of the reinforcement suture relative to the surgical locking button.
